(19) United States
(12) Patent Application Publication
(10) Pub. No.: US 2024/0285173 A1
(43) Pub. Date: Aug. 29, 2024

(54) BODY TEMPERATURE ESTIMATING DEVICE, BODY TEMPERATURE ESTIMATING METHOD AND BODY TEMPERATURE ESTIMATING SYSTEM

(71) Applicant: NIPPON TELEGRAPH AND TELEPHONE CORPORATION, Tokyo (JP)

(72) Inventors: Shin TOYOTA, Musashino-shi (JP); Kazuyoshi ONO, Musashino-shi (JP)

(73) Assignee: NIPPON TELEGRAPH AND TELEPHONE CORPORATION, Tokyo (JP)

(21) Appl. No.: 18/570,158

(22) PCT Filed: Jul. 5, 2021
(86) PCT No.: PCT/JP2021/025280

§ 371 Date: Dec. 14, 2023

(51) Int. Cl. A61B 5/01

(52) U.S. Cl. CPC A61B 5/01; A61B 2503/40

(57) ABSTRACT

A body temperature estimation device includes a measurement unit that measures a temperature of an eye of an animal, and a body temperature estimation unit that estimates a core body temperature of the animal by setting the temperature measured by the measurement unit in a body temperature estimation model and simulating heat conduction.

### TECHNICAL FIELD

The present invention relates to a body temperature estimation device (a body temperature estimating device), a body temperature estimation method (a body temperature estimating method), and a body temperature estimation system (a body temperature estimating system).

### BACKGROUND ART

A body temperature estimation model for estimating a body temperature such as a core body temperature of a human using a simulation is used for evaluation of an outdoor environment and verification of thermal physiology of a human body (for example, Non Patent Literature 1). Also, there is a demand for a wearable device capable of preventing heat stroke and detecting infection by obtaining a temperature change in real time (for example, Non Patent Literature 2).

### CITATION LIST

### Non Patent Literature

Non Patent Literature 1: Shinichi Tanabe, Junta Nakano, and Kozo Kobayashi: Development of 65-node Thermoregulation-Model for Evaluation of Thermal Environment, Transactions of AIJ. Journal of Architecture, Planning and Environmental Engineering (541), 9-16, 2001
Non Patent Literature 2: Fiscal Year 2012 Study on Heat Island Effects and Countermeasures after the Great East Japan Earthquake, Reference Material 1-5 “Results of Investigation on Human Body Thermophysiological Model”

### SUMMARY OF INVENTION

### Technical Problem

However, for example, since the model described in Non Patent Literature 1 aims to be able to express a temperature change of a distal portion of a body such as a fingertip and a state of keeping a head cool and keeping feet warm, the model is over-engineered for obtaining a body temperature change in a resting state, and the computational complexity may increase. Also, since there are a plurality of parameters required for the simulation and it is necessary to attach sensors to the entire body, it is difficult to perform the simulation with a wearable device.

The present invention provides a body temperature estimation device that can perform body temperature estimation more easily.

### Solution to Problem

An aspect of the present invention is a body temperature estimation device including a measurement unit that measures a temperature of an eye of an animal, and a body temperature estimation unit that estimates a core body temperature of the animal by setting the temperature measured by the measurement unit in a body temperature estimation model and simulating heat conduction.

### Advantageous Effects of Invention

The body temperature estimation device according to the present invention can perform body temperature estimation more easily.

### BRIEF DESCRIPTION OF DRAWINGS

FIG. 1 is a diagram illustrating a configuration example of a body temperature estimation device 1.

FIG. 2 is a flowchart illustrating an operation of the body temperature estimation device 1.

FIG. 3A is a front view of a body temperature estimation model.

FIG. 3B is a side view of the body temperature estimation model.

FIG. 4 is a diagram illustrating a configuration of a body temperature estimation system 3.

### DESCRIPTION OF EMBODIMENTS

Hereinbelow, embodiments of the present invention will be described in detail with reference to the drawings.

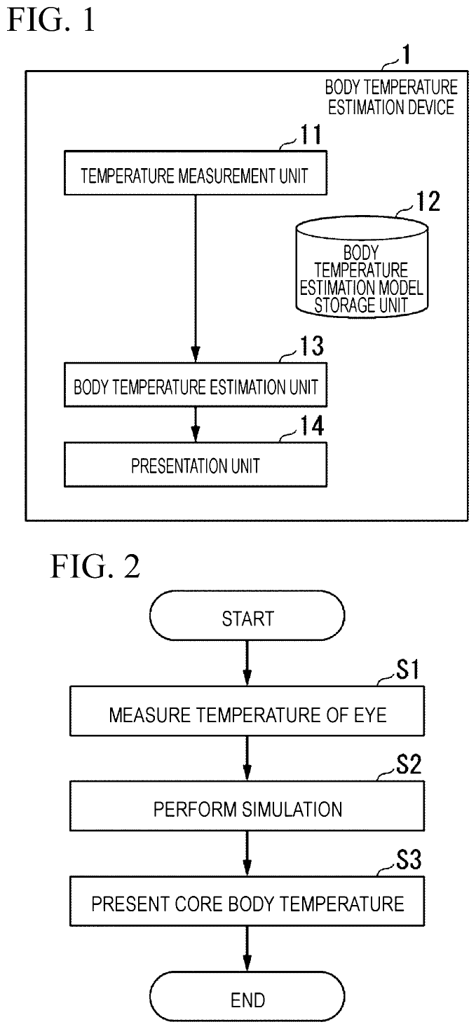

FIG. 1 is a diagram illustrating a configuration example of a body temperature estimation device 1. The body temperature estimation device 1 estimates the body temperature by measuring the temperature of the lacrimal caruncle. The body temperature estimation device 1 includes a temperature measurement unit 11, a body temperature estimation model storage unit 12, a body temperature estimation unit 13, and a presentation unit 14.

The temperature measurement unit 11 measures the temperature of the eye of a user. The temperature measurement unit 11 may measure, for example, the temperature of the lacrimal caruncle portion of the eye of the user. Arteries and veins run in parallel near the lacrimal caruncle, and the temperature (core body temperature) of the hypothalamus is easily transferred by heat transport through the blood vessels. Also, in terms of the body temperature of a human in a resting state, since it can be considered that the heat transfer from the head to the body and the heat transfer from the body to the head are in an equilibrium state, it can be assumed that the temperature of the hypothalamus and the temperature of the body are equal. The temperature measurement unit 11 estimates the temperature distribution of the entire face by means of, for example, a thermal camera, and sets the highest temperature as the temperature of the lacrimal caruncle portion. The temperature measurement unit 11 estimates the temperature distribution of the entire face by means of, for example, a thermal camera, specifies the lacrimal caruncle portion by means of face recognition, and estimates the temperature of the lacrimal caruncle portion. The temperature measurement unit 11 may measure the temperature of the lacrimal caruncle portion of the eye of the user by bringing a thermocouple type temperature sensor into contact with the lacrimal caruncle portion.

The body temperature estimation model storage unit 12 stores a body temperature estimation model. The body temperature estimation model is a model used by the body temperature estimation unit 13 for simulation. Details of the body temperature estimation model will be described below.

The body temperature estimation unit 13 estimates the core body temperature of the user by setting the temperature measured by the temperature measurement unit 11 in the body temperature estimation model stored in the body temperature estimation model storage unit 12 and performing simulation.

The presentation unit 14 presents the core body temperature of the user estimated by the body temperature estimation unit 13. The presentation unit 14 outputs the estimated core body temperature of the user to the outside by displaying the estimated core body temperature on a display, for example.

FIG. 2 is a flowchart illustrating an operation of the body temperature estimation device 1.

First, the temperature measurement unit 11 measures the temperature of the eye of the user (step S1). The body temperature estimation unit 13 sets the temperature measured by the temperature measurement unit 11 in the body temperature estimation model and performs simulation (step S2). The presentation unit 14 presents the core body temperature of the user estimated by the body temperature estimation unit 13 (step S3).

The body temperature estimation model is created by dividing the body into several nodes. The nodes are nodes divided to correspond to actual human head portions such as the brain and the eye. In general, the smaller the number of nodes, the further the computational complexity of simulation decreases. Also, the body temperature estimation model includes a node of a blood vessel connecting the brain and the eye.

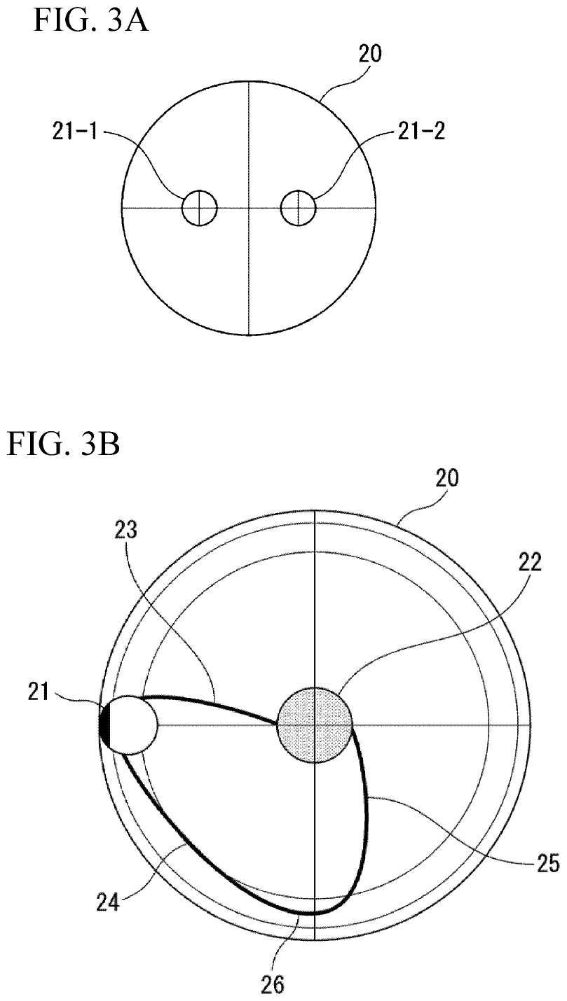

FIGS. 3A and 3B illustrate an example of the body temperature estimation model. FIG. 3A is a front view of the body temperature estimation model, and FIG. 3B is a side view of the body temperature estimation model. In the body temperature estimation model illustrated in FIGS. 3A and 3B, the number of nodes representing organs is seven (a hypothalamus, a brain, cerebrospinal fluid, a skull, skin, an eyeball, and a blood vessel). In addition, assumed as a method in which heat is transferred from the hypothalamus to the lacrimal caruncle are heat transfer by the tissue in the brain and heat transport through the blood vessels, and heat transport through the blood vessels is considered to have a higher contribution. Therefore, a blood vessel model is incorporated in the body temperature estimation model.

FIG. 3A illustrates a virtual sphere 20, a right eyeball 21-1, and a left eyeball 21-2. In FIG. 3B, a hypothalamus 22, an ophthalmic artery 23, an angular artery 24, an internal carotid artery 25, and an external carotid artery 26 are illustrated. The body temperature estimation model is created in the following procedure. First, the hypothalamus 22 is disposed as a heat source at the center of the virtual sphere 20. Thereafter, the brain, the cerebrospinal fluid, the skull, and the skin are sequentially disposed on the outer periphery of the hypothalamus 22. Thereafter, the eyeballs 21 are created at the outer edge of the skin so as to be bilaterally symmetrical. At this time, the thermal conductivity, density, and specific heat of each portion are set.

Thereafter, the ophthalmic artery 23, the angular artery 24, the internal carotid artery 25, and the external carotid artery 26 are disposed to simulate heat transport by the blood from the hypothalamus 22 to the lacrimal caruncle of the eye. The ophthalmic artery 23 is an artery that branches from the internal carotid artery 25 and extends to the eyeball 21 along the optic nerve connecting the eyeball 21 and the hypothalamus 22. The angular artery 24 is an artery that extends from the external carotid artery 26 along the skull to the eyeball 21. The internal carotid artery 25 extends from the lower part of the virtual sphere 20 through the brain to the vicinity of the hypothalamus 22. The external carotid artery 26 extends from the lower part of the virtual sphere 20 through the structure inside the skull and the brain to the vicinity of the hypothalamus 22. The internal carotid artery 25 anastomoses with the ophthalmic artery 23 in the vicinity of the hypothalamus 22, and the ophthalmic artery 23 anastomoses with the angular artery 24 in the eyeball 21. Also, the mass flow rate and specific heat of the blood are set. In order to simulate the release of heat from the angular artery 24 to the outside of the body, the characteristics of the outside air (for example, the temperature of the outside air) are set. The internal carotid artery 25 and the external carotid artery 26 are connected to the torso in thermal equilibrium with the head. That is, in the present model, the hypothalamus 22, the internal carotid artery 25, and the external carotid artery 26 are treated as being in a thermal equilibrium state and having no change in the thermodynamic state quantity. In another embodiment, the internal carotid artery 25 and the external carotid artery 26 may be treated as one model without being distinguished from each other. Then, each node of the eyeball 21, the hypothalamus 22, the ophthalmic artery 23, the angular artery 24, the internal carotid artery 25, and the external carotid artery 26 is divided into a plurality of meshes that are minimum units of simulation.

The body temperature estimation unit estimates the core body temperature from the measured temperature of the eye by simulating heat conduction to each organ by means of blood using the aforementioned body temperature estimation model. That is, the body temperature estimation unit 13 simulates heat transport from the internal carotid artery 25 in thermal equilibrium with the hypothalamus 22 via the ophthalmic artery 23 to the lacrimal caruncle and heat transport from the external carotid artery 26 in thermal equilibrium with the hypothalamus 22 via the angular artery 24 to the lacrimal caruncle. As a simulation method, a finite element method can be used. Then, the body temperature estimation unit derives the temperature of the hypothalamus, which is the heat source, so that the temperature of the lacrimal caruncle in the body temperature estimation model becomes the measured temperature. For example, the body temperature estimation unit sets the temperature of the hypothalamus in the body temperature estimation model, performs simulation of heat transport, and based on the temperature of the eye obtained as a result of the simulation, searches for the temperature of the hypothalamus in the body temperature estimation model using the gradient method.

In this manner, the body temperature estimation device 1 according to the embodiment includes the measurement unit that measures the temperature of the eye of an animal, and the body temperature estimation unit that estimates the core body temperature of the animal by setting the temperature measured by the measurement unit in the body temperature estimation model and simulating heat conduction. As a result, the body temperature estimation device 1 can measure the core body temperature easily and accurately.

Hereinbelow, an example of the present embodiment will be described.

In the present example, COMSOL (registered trademark) was used as simulation software. In addition, the heat conduction in the body temperature estimation model was based on Pennes' bio-heat transfer equation shown in Equation (1).

$$k \frac{{\partial }^{2} T}{\partial {x}^{2}} = {ρ}_{t} {c}_{t} ( \frac{\partial T}{\partial t} ) + {W}_{b} {c}_{b} ( T - {T}_{a} ) \qquad ( 1 )$$

Here, k represents the thermal conductivity of the tissue, T represents the temperature of the tissue, ρₜ represents the density of the tissue, cₜ represents the specific heat of the tissue, W_{b} represents the mass flow rate of the blood per unit volume of the tissue, c_{b} represents the specific heat of the blood, and Tₐ represents the temperature of the artery.

When the temperature of the eye was set in the body temperature estimation model and heat conduction was simulated using COMSOL based on Pennes' bio-heat transfer equation, the temperature of each portion was output, and the temperature of the hypothalamus 22 was set as an estimated value of the core body temperature. In the present example, the total number of meshes was 43,778 when COMSOL was used. This amount of data to be processed can easily be processed even in a wearable device such as a smartphone.

### Modification Example 1

A body temperature estimation equation may be derived with use of the body temperature estimation model in advance. The body temperature estimation equation is an equation indicating the relationship between the temperature of the eye and the core body temperature of the human. The body temperature estimation equation can be obtained, for example, by preparing several sets of combinations of the eye temperature and the core body temperature estimated when the eye temperature is set in the body temperature estimation model, and applying the least squares method to the combinations. By substituting the eye temperature into the body temperature estimation equation, the estimated value of the core body temperature can be calculated. Similarly, the body temperature estimation equation can be derived in a case of measuring the temperature of the lacrimal caruncle.

Furthermore, the body temperature estimation unit 13 may estimate the body temperature using the body temperature estimation equation instead of the body temperature estimation model. The body temperature estimation unit 13 calculates an estimated value of the temperature of the hypothalamus by substituting the temperature of the lacrimal caruncle portion measured by the temperature measurement unit 11 into the body temperature estimation equation.

### Modification Example 2

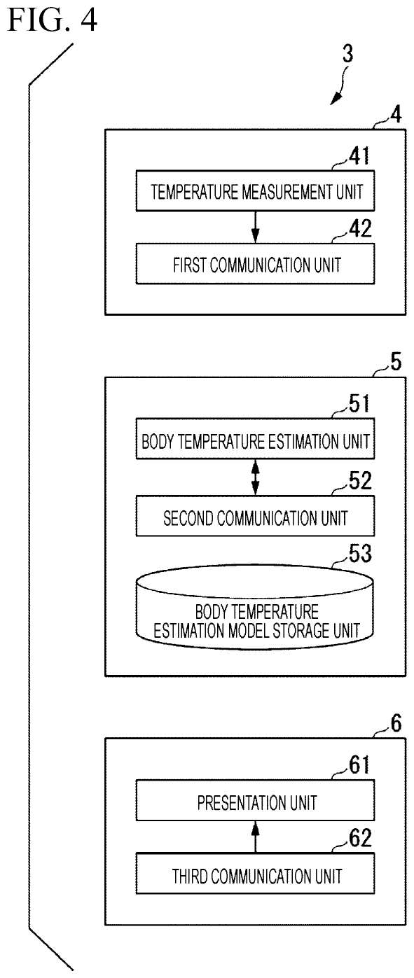

The temperature measurement unit 11, the body temperature estimation unit 13, and the presentation unit 14 may be provided by separate devices. FIG. 4 is a diagram illustrating a configuration of a body temperature estimation system 3. The body temperature estimation system 3 includes a temperature measurement device 4, a body temperature estimation device 5, and a presentation device 6.

The temperature measurement device 4 includes a temperature measurement unit 41 and a first communication unit 42. The body temperature estimation device 5 includes a body temperature estimation unit 51, a second communication unit 52, and a body temperature estimation model storage unit 53. The presentation device 6 includes a presentation unit 61 and a third communication unit 62.

The temperature measurement unit 41, the body temperature estimation unit 51, the body temperature estimation model storage unit 53, and the presentation unit 61 have equivalent functions to those of the temperature measurement unit 11, the body temperature estimation unit 13, the body temperature estimation model storage unit 12, and the presentation unit 14, respectively.

Information among the temperature measurement device 4, the body temperature estimation device 5, and the presentation device 6 is transmitted and received by the first communication unit 42, the second communication unit 52, and the third communication unit 62.

Although an embodiment of the present invention has been described in detail with reference to the drawings, specific configurations are not limited to those in the embodiment, and include designs and the like without departing from the scope of the invention.

The subject whose body temperature is estimated by the body temperature estimation device 1 is not limited to a human (human), and for example, a body temperature estimation model may be created for an animal (particularly, a vertebrate) other than a human to estimate the body temperature of the animal. In the present specification, “animal” includes human.

### REFERENCE SIGNS LIST

1 Body temperature estimation device
11, 41 Temperature measurement unit
12, 53 Body temperature estimation model storage unit
13, 51 Body temperature estimation unit
14, 61 Presentation unit
20 Virtual sphere
21 Eyeball
22 Hypothalamus
23 Ophthalmic artery
24 Angular artery
25 Internal carotid artery
26 External carotid artery
3 Body temperature estimation system
4 Temperature measurement device
5 Body temperature estimation device
6 Presentation device
42 First communication unit
52 Second communication unit
62 Third communication unit

1. A body temperature estimation device comprising:
a measurement unit that measures a temperature of an eye of an animal; and
a body temperature estimation unit that estimates a core body temperature of the animal by setting the temperature measured by the measurement unit in a body temperature estimation model and simulating heat conduction.

2. The body temperature estimation device according to claim 1, wherein the measurement unit measures a temperature of a lacrimal caruncle portion of the eye of the animal.

3. The body temperature estimation device according to claim 1, wherein the body temperature estimation model includes a blood vessel connecting the eye and a brain and a model of an organ covering the blood vessel, and wherein the body temperature estimation unit simulates heat transport by blood passing through the blood vessel.

4. A body temperature estimation device comprising:
a measurement unit that measures a temperature of an eye of an animal; and
a body temperature estimation unit that estimates a core body temperature of the animal by inputting the temperature measured by the measurement unit in a body temperature estimation equation indicating a relationship between a temperature of an eye of the animal and a core body temperature of the animal.

5. A body temperature estimation method comprising:
a measurement step of measuring a temperature of an eye of an animal; and
a body temperature estimation step of estimating a core body temperature of the animal by setting the temperature measured in the measurement step in a body temperature estimation model and simulating heat conduction.

6. A body temperature estimation system comprising:
a temperature measurement device that measures a temperature of an eye of an animal; and
a body temperature estimation device that estimates a core body temperature of the animal by setting the temperature measured by the temperature measurement device in a body temperature estimation model and simulating heat conduction.
